(19) United States
(12) Patent Application Publication
(10) Pub. No.: US 2018/0246104 A1
(43) Pub. Date: Aug. 30, 2018

(54) METHOD FOR DETECTING CIRCULATING TUMOR CELLS AND USES THEREOF

(71) Applicant: AGENCY FOR SCIENCE, TECHNOLOGY AND RESEARCH, Singapore (SG)

(72) Inventor: Say Li KONG, Singapore (SG)

(21) Appl. No.: 15/753,301

(22) PCT Filed: Aug. 18, 2016
(86) PCT No.: PCT/SG2016/050399

§ 371 Date: Feb. 17, 2018

(30) Foreign Application Priority Data
Aug. 18, 2015 (SG) 10201506501X

(51) Int. Cl. G01N 33/574

(52) U.S. Cl. CPC G01N 33/5748

(57) ABSTRACT

A method for the detection and/or diagnosis of cancer in a patient is disclosed. The method involves detecting circulating tumor cells (CTCs) in a sample obtained from the patient using an antibody specifically binding to an antigen in a protein expressed by the CTCs, wherein the antigen comprises a mutation and wherein the mutation renders the patient receptive or resistance to an anti-cancer drug. Claimed methods include the use of an antibody specific for the EGFR L858R or EGFR exon 19 (ΔE746-A750) mutations for detecting lung cancers and the use of an antibody specific for the K-Ras G12V, K-Ras G12C, K-Ras G12S, K-Ras G12D, or K-Ras G13D mutations for detecting colorectal cancers.

### CROSS-REFERENCE TO RELATED APPLICATIONS

This application claims the benefit of priority of Singapore provisional application No. 10201506501X, filed on 18 Aug. 2015, the contents of which are being hereby incorporated by reference in their entirety for all purposes.

### FIELD OF THE INVENTION

The present invention relates to the field of biochemistry. Particularly, the present invention relates to the method of detecting tumor cells, more particularly circulating tumor cells (CTCs) of interest. The present invention also relates to use of the method of detecting CTCs in the detection and/or diagnosis and treatment of cancer.

### BACKGROUND OF THE INVENTION

Cancer is the second leading cause of death worldwide, accounting for 8.2 million deaths in 2012. Cancer mortality can be significantly reduced if detected and treated early. In addition, during cancer treatment, it is essential to monitor treatment efficacy and cancer progression in order to determine the suitable treatment regimen for the patient. Methods for reliable detection of cancer and the monitor of cancer progression mainly involve the use of endoscopies, radioactive scannings and tissue biopsy, which are expensive and invasive procedures that impose certain health risks to the patient. Hence there is a need to provide a non-invasive method for effectively detecting cancer and monitoring cancer progression in a patient.

CTCs are cells shed from the primary tumor into the blood stream or the lymphatic system, which can then circulate to and invade other organs. CTCs found in the blood of cancer patients can be useful predictors for the detection of cancer and the monitoring of cancer progression. CTCs are particularly useful in the detection and monitoring of metastatic progression. However, the detection of CTCs remains as a technical challenge that hinders the use of CTCs for the diagnosis or prognosis of cancers. The current technology for CTC enumeration is largely relied on the physical or biological properties of CTCs that enrich for larger CTCs or CTCs that express epithelial markers such as EPCAM or HER2. However, the detection of CTCs based on the expression of these epithelial markers is inaccurate as some cells that over-express these epithelial markers are benign epithelial cells which lack clinical utility value. In addition, certain CTCs, such as CTCs from lung cancers, have been demonstrated to have low or no expression of epithelial markers, thus hinder the detection of such CTCs. Moreover, tumor cells generally undergo epithelial-to-mesenchymal transition (EMT) when shedding from the primary tumor, thus the current technology that relies on the epithelial markers will generally under-estimate the presence of CTCs with mesenchymal phenotype (i.e. false negative results).

Further, the existing technologies for the detection of CTCs generally involve DNA extraction, amplification and sequencing, which are laborious and time-consuming. These techniques counteract with the clinical needs for tests with short turnaround time.

Therefore, there is a need to provide a method for the detection and treatment of cancer, and for the monitoring of cancer progression and treatment efficacy, that involves efficient defection of CTCs, which overcome, or at least ameliorate, one or more of the disadvantages described above.

### SUMMARY OF THE INVENTION

In one aspect of the present invention, there is provided a method of detecting and/or diagnosing cancer in a patient, wherein the method comprises detecting circulating tumor cells (CTCs) in a sample obtained from the patient using an antibody specifically binding to an antigen in a protein expressed by the CTCs, wherein the antigen comprises a mutation and wherein the mutation renders the patient receptive or resistant to an anti-cancer treatment.

In another aspect of the present invention, there is provided a method of treating a cancer in a patient, wherein the method comprises: detecting circulating tumor cells (CTCs) in a sample obtained from the patient using an antibody specifically binding to an antigen in a protein expressed by the CTCs, wherein the antigen comprises a mutation and wherein the mutation renders the patient receptive or resistant to an anti-cancer treatment; and treating the patient with the anti-cancer treatment suitable for the cancer to be treated in light of the results in (a).

In a further aspect of the present invention, there is provided a method of detecting and/or diagnosing lung cancer in a patient, wherein the method comprises detecting circulating tumor cells (CTCs) in a sample obtained from a patient using an antibody specifically binding to an antigen in a protein expressed by the CTCs, wherein the antigen comprises a mutation of EGFR L858R or EGFR exon 19 (ΔE746-A750), and wherein the mutation renders the patient receptive to the treatment with a tyrosine kinase inhibitor. In yet a further aspect of the present invention, there is provided a method of detecting and/or diagnosing colorectal cancer in a patient, wherein the method comprises detecting circulating tumor cells (CTCs) in a sample obtained from a patient using an antibody specifically binding to an antigen in a protein expressed by the CTCs, wherein the antigen comprises a mutation of K-Ras selected from the group consisting of K-Ras G12V, K-Ras G12C, K-Ras G12S, K-Ras G12D and K-Ras G13D, and wherein the mutation renders the patient resistant to the treatment with a monoclonal antibody against EGFR.

### BRIEF DESCRIPTION OF THE DRAWINGS

The invention will be better understood with reference to the detailed description when considered in conjunction with the non-limiting examples and the accompanying drawings, in which:

FIG. 1A shows the Western blot results of the evaluation of the specificity of the EGFR L858R and EGFR exon19 (ΔE746-A750) deletion antibodies using samples of the H1975 and PC9 cancer cell lines. H1975 cell line is known as positive for EGFR L858R and negative for EGFR exon19 (ΔE746-A750), while PC9 cancer cell line is known as positive for EGFR exon19 (ΔE746-A750) and negative for EGFR L858R. Lane 1 is the result of EGFR-L858R for H1975, lane 2 is the result of EGFR exon19 (ΔE746-A750) for H1975, Lane 3 is the result of EGFR-L858R for PC9, lane 4 is the result of EGFR exon19 (ΔE746-A750) for PC9. Lane 5 and 6 serve as the loading control (β-actin) for H1975, and lane 7 and 8 serve as the loading control (β-actin) for PC9. FIG. 1B shows the Western blot results of the evaluation of the specificity of the KRAS-G12V antibody using samples of the SW620 colorectal cancer cells and the PC9 lung cancer cells. SW620 cell line is known as positive for KRAS-G12V, while PC9 cancer cell line is known as negative for KRAS-G12V. Lane 9 is the result of KRAS-G12V for SW620, lane 10 is the result of KRAS-G12V for PC9. Lane 11 serves as the loading control (β-actin) for SW620, and lane 12 serves as the loading control (β-actin) for PC9. The results in FIGS. 1A and 1B show that signal was detected only in samples that carried the correct mutation signatures, indicating the good specificity of the antibodies (lanes 1 to 4, lanes 9 and 10).

FIG. 2 shows the immuno-fluorescent results of PC9 cells, H1975 cells and a 50%/50% mixture of PC9 and H1975 cells using EGFR exon19 (ΔE746-A750) antibody (Panel A) and EGFR L858R (Panel B). As shown in Panel A, positive signal for EGFR exon19 (ΔE746-A750) antibody was only observed in PC9 cells; while as shown in Panel B, positive signal for EGFR L858R antibody was only observed in H1975. These results further confirmed the specificity of the EGFR exon19 (ΔE746-A750) and EGFR L858R antibodies.

FIG. 3 shows the immuno-fluorescent results of 100% SW620 cells using KRAS-G12V antibody. As shown, positive signals for KRAS-G12V were observed in all SW620 cells, confirming the sensitivity of the KRAS-G12V antibody.

FIG. 4 shows the immuno-fluorescent results of blood samples from healthy subjects spiked with cancer cell lines H1975 and PC9 with known mutation signature. CD45 antibody was added to the samples in order to distinguish the white blood cells (CD45 +ve) in the blood samples from the spiked cancer cells (CD45 −ve). Panel A shows blood sample spiked with H1975 cells being detected using EGFR L858R antibody (marked by arrow) and Panel B shows blood sample spiked with PC9 cells being detected using EGFR exon19 (ΔE746-A750) antibody (marked by arrow).

FIG. 5 shows the immuno-fluorescent results of blood samples from lung cancer patients with positive EGFR L858R mutation in the primary tumor. Panel A shows an example that is positive for EGFR L858R, indicating the presence of CTCs, and Panel B shows an example that is negative for EGFR L858R, indicating the absence of CTCs.

FIG. 6 shows the immuno-fluorescent results of blood samples obtained from healthy subjects. As shown, the cells in the blood samples obtained from healthy subjects are negative for EGFR L858R signals, indicating the absence of CTCs.

FIG. 7 is a representation of epidermal growth factor receptor (EGFR) showing the distribution of exons in the extracellular domain (EGF binding), transmembrane domain (TM) and intracellular domain (comprising the tyrosine kinase and autophosphorylation regions). Exons 18-21 in the tyrosine kinase region where the relevant mutations are located are expanded, and a detailed list of EGFR mutations in these exons that are associated with sensitivity or resistance to tyrosine kinase inhibitor is shown.

FIG. 8 illustrates an application of a method of the present disclosure for the selection of personalized treatment. In this example, a cancer patient carries a primary tumor with mutation profile A (MtA) and mutation profile B (MtB). The detection of CTCs using a method of the present disclosure indicates a higher level of MtA as a consequence of being the predominant tumor type, and a first tumor therapy is thus selected to target MtA first. After treatment, the level of MtA decreased, and subsequently, a second tumor therapy is selected to target MtB. After this personalized course of treatment using the combination of the first and second tumor therapies, the person has a better chance of survival as the primary tumor carrying both types of mutations are prevented from metastasizing.

### DETAILED DESCRIPTION OF THE PRESENT INVENTION

The present disclosure provides a method of detecting and/or diagnosing cancer in a patient. Thus, in one aspect, there is provided a method of detecting and/or diagnosing cancer in a patient, wherein the method comprises detecting circulating tumor cells (CTCs) in a sample obtained from the patient using an antibody specifically binding to an antigen in a protein expressed by the CTCs, wherein the antigen comprises a mutation and wherein the mutation renders the patient receptive or resistant to the treatment with an anti-cancer drug.

An antigen comprising a mutation refers to a mutation in the amino acid sequence of the protein, wherein the mutated amino acid(s) is/are the antigen to which the antibody binds or forms at least part of the amino acid sequence to which the antibody binds. The term “diagnosis” or “diagnosing” or other grammatically variants thereof as used herein refers to detecting a disease or determining the status or degree of a disease. More specifically, the diagnosis of cancer is based on the results from the detection of CTCs performed using a method as disclosed in the present application. In one example, the method of diagnosis does not include the step of evaluating the results and making a decision that is generally carried out by a medical practitioner (e.g. a doctor, a nurse, or a veterinary).

The term “circulating tumor cells” (CTCs) used herein refers to cells that have shed into the vasculature or lymphatics from a primary tumor and can be carried around the body in the circulation. CTCs can lead into the growth of additional tumors in distant organs, resulting in metastases. In general, CTCs are cancer cells with an intact, viable nucleus. They express cytokeratins which demonstrates the epithelial origin of the CTCs. They also have an absence of CD45 protein indicating the cell is not of hematopoietic origin. CTCs are often larger cells with irregularity shape or subcellular morphology, and they can be present as clusters of two or more individual CTCs. Usually these clusters are associated with increased metastatic risk and poor prognosis.

The current technology for CTC enumeration is largely relied on the physical or biological properties of CTCs that enrich for larger CTCs or CTCs that express epithelial markers such as EPCAM or HER2. A further issue with the current technologies used for the detection of CTCs is that they could not provide any information useful for the selection of cancer therapies or for the provision of personalized treatment to cancer patients. The methods of the present disclosure, which detect CTCs based on mutations that render the patients receptive or resistant to an anti-cancer treatment, make the selection of cancer therapies and the provision of personalized cancer treatment possible.

In some examples, the presence of CTCs in the sample obtained from the patient indicates that the patient has cancer. In some examples, it is considered that the CTCs are “present” if it is detectable above the background noise of the respective detection method used (e.g., 2-fold, 3-fold, 5-fold, or 10-fold higher than the background; e.g., 2-fold or 3-fold over background).

The term “sample” used herein refers to a biological sample, or a sample that comprises at least some biological materials such as cells. The biological samples of this disclosure may be any sample suspected to contain CTCs, including solid tissue samples, such as bone marrow, and liquid samples, such as whole blood, blood serum, blood plasma, cerebrospinal fluid, central spinal fluid, lymph fluid, cystic fluid, sputum, stool, pleural effusion, mucus, pleural fluid, ascitic fluid, amniotic fluid, peritoneal fluid, saliva, bronchial washes and urine. In some examples, the biological sample is not a tissue sample or not a sample obtained from tissue biopsy. In some examples, the biological sample is a liquid sample. In one specific example, the biological sample is a blood sample. As will be appreciated by those skilled in the art, a biological sample can include any fraction or component of blood, without limitation, T-cells, monocytes, neutrophiles, erythrocytes, platelets and microvesicles such as exosomes and exosome-like vesicles. CTCs can be isolated from a biological sample, such as a whole blood sample, using any method known to those of skill in the art.

Since the methods of the present disclosure can detect CTCs using a liquid sample, one advantage of such methods is that no invasive tissue biopsy is required.

The biological samples may be obtained from any organism, including mammals such as humans, primates (including but not limited to monkeys, chimpanzees, orangutans, and gorillas), domestic animals (including but not limited to cats, dogs, and rabbits), farm animals (including but not limited to cows, horses, goats, sheep, and pigs), and rodents (including but not limited to mice, rats, hamsters, and guinea pigs). In a specific example, the biological sample is obtained from human.

It is noted that, as used herein, the terms “organism,” “individual,” “subject,” or “patient” are used as synonyms and interchangeably.

The organism may be a healthy organism or an organism that suffers from a disease condition. Disease conditions may include any disease. In some examples, the disease is cancer, diabetes, metabolic syndrome, or an autoimmune disorder. In some examples, the healthy or diseased organism is a human organism. In one example, the biological sample is obtained from a healthy organism for the detection of cancer. In some examples, the biological sample is obtained from a patient suffering from cancer to determine the status of the cancer, such as the stage or progression of the cancer, or the effect of anti-cancer treatment on the cancer. In some examples, the biological sample is obtained from a non-cancer patient with another known disease, to detect if the patient has also developed cancer. In some other examples, the biological sample is obtained from a patient known to have cancer and another disease, to determine the status of the cancer, such as the stage or progression of the cancer, or the effect of anti-cancer treatment on the cancer. In some examples, the healthy or diseased organism is an animal model for a disease condition, such as cancer. A person of ordinary skill understands that animal models for various disease conditions are well known in the art.

A diseased organism known to have cancer may be untreated or may have received treatment, such as chemotherapy, radiotherapy, surgical treatment and immunotherapy. The treatment may predate the sample collection or be ongoing at the time of sample collection.

The samples of this disclosure may each contain a plurality of cell populations and cell subpopulations that can be distinguishable by methods well known in the art (e.g., FACS, immunohistochemistry). For example, a blood sample may contain populations of non-nucleated cells, such as erythrocytes or platelets, and populations of nucleated cells such as white blood cells (WBCs) and CTCs. WBCs may contain cellular subpopulations such as neutrophils, lymphocytes, monocytes, eosinophils, basophils and the like. The samples of this disclosure may be enriched or non-enriched samples, i.e., they are enriched or not enriched for any specific population or subpopulation of nucleated or non-nucleated cells. The WBCs in the samples could be distinguished from the CTCs using WBC markers. One example of WBC marker is CD45 (CD stands for cluster of differentiation).

The term “antibody” as used herein means an immunoglobulin molecule able to bind to a specific epitope on an antigen. Antibodies can be comprised of a polyclonal mixture, or may be a monoclonal antibody. Further, antibodies can be entire immunoglobulins derived from natural sources, or from recombinant sources. The antibodies used in the methods described herein may exist in a variety of forms, including for example as a whole antibody, or as an antibody fragment, or other immunologically active fragment thereof, such as complementarity determining regions. Similarly, the antibody may exist as an antibody fragment having functional antigen-binding domains, that is, heavy and light chain variable domains. Also, the antibody fragment may exist in a form selected from the group consisting of, but not limited to: Fv, Fab, F(ab)2, scFv (single chain Fv), dAb (single domain antibody), bi-specific antibodies, diabodies and triabodies.

An antibody can have one or more binding sites. If there is more than one binding site, the binding sites can be identical to one another or can be different.

In one example, the antibodies used in the methods described herein are capable of specific binding to an antigen in a protein expressed by the CTCs.

The term “specific binding” or “binding” and their grammatical variants refer to the binding of an antibody to its target antigen.

The term “antigen” refers to a molecule or a portion of a molecule capable of being bound by an antibody or immunological functional fragment thereof. In some examples, an antigen can possess one or more epitopes. In a specific example, an antigen only possesses one epitope. The term “epitope” means the amino acids of a target molecule that are contacted by an antibody when the antibody is bound to the antigen. The term “epitope” includes any subset of the complete list of amino acids of the antigen that are contacted when an antibody is bound to the antigen. The 3-dimensional structure of the epitope is generally determined by the amino acid sequence of the antigen, and a mutation in the amino acid sequence of the antigen can change the 3-dimensional structure of the epitope, thereby allowing the antigen to be recognized and bound by an antibody that is specific to the mutation in the amino acid sequence of the antigen. In some examples of the present disclosure, a mutation in the amino acid sequence of a protein expressed by the CTCs results in a specific 3-dimensional structure of the epitope that allows the protein to be bound by an antibody. In some examples, an antigen comprises one or more epitopes each carrying one or more mutations in the amino acid sequences. In some other examples, a protein expressed by the CTCs comprises one or more antigens comprising one or more epitopes each carrying one or more mutations in the amino acid sequences.

Specific examples of antibodies that can be used in the methods of the present disclosure include but are not limited to: EGFR (E746-A750del Specific) (D6B6) XP® Rabbit mAb (Cell Signaling Technology, Inc.); EGFR (L858R Mutant Specific) (43B2) Rabbit mAb (Cell Signaling Technology, Inc.); B-Raf (V600E) Mouse mAb (NewEast Biosciences); B-Raf (V600K) Mouse mAb (NewEast Biosciences); Ras (G12V Mutant Specific) (D2H12) Rabbit mAb (Cell Signaling Technology, Inc.); Ras (G12D Mutant Specific) (D8H7) Rabbit mAb (Cell Signaling Technology, Inc.); Ras (G13D) Mouse mAb (NewEast Biosciences); Ras (G12S) Mouse mAb (NewEast Biosciences); EML4-ALK Mouse mAb (NewEast Biosciences); KIT (L576P) Mouse mAb (NewEast Biosciences); KIT (W557R) Mouse mAb (NewEast Biosciences); and KIT (V559D) Mouse mAb (NewEast Biosciences).

In one example, the antibody is coupled to a detectable label by methods known in the art, such as direct antibody conjugation and indirect antibody conjugation. The term “direct antibody conjugation” refers to the conjugation of the primary antibody to a detectable label. The term “indirect antibody conjugation” refers to a two-step method wherein the primary antibody is not conjugated to a detectable label. A secondary antibody directed against the primary antibody is used, wherein the secondary antibody is conjugated to a detectable label. The detectable label can be any one of the following: a fluorescent group, a radioisotope, a stable isotope, an enzymatic group, a chemiluminescent group or a biotinyl group.

In one specific example, an antibody that specifically binds to an antigen in a protein expressed by the CTCs is used as a primary antibody. A sample to be tested for the presence of CTCs is incubated with the primary antibody to allow the antibody to bind to the antigen in a protein expressed by the CTCs. After incubation, the unbound primary antibody is removed from the sample, and the sample is subsequently incubated with a secondary antibody carrying a detectable label to allow the secondary antibody to bind to the primary antibody. Thereafter, the unbound secondary antibody is removed from the sample, and the detectable label carried by the secondary antibody is identified to detect the CTCs.

A number of other methods are known in the art for detecting binding of an antibody to its antigen in an immunoassay and are within the scope of the present disclosure. Examples of such methods include but are not limited to, immunofluorescence, immunohistochemistry, immunoassays such as Western blots, enzyme-linked immunosorbant assay (ELISA), immunoprecipitation, radioimmunoassay, dot blotting, Fluorescence-activated cell sorting (FACS) and mass cytometry.

The term “mutation” as used herein refers to tumor-associated mutations, which are mutations in nucleic acid sequences that affect development of a tumor in a subject. A tumor-associated mutation can activate cellular proliferation, thus leading to emergence of a malignant tumor or escalation of tumor growth. A tumor-associated mutation can confer properties on a tumor that facilitate its spread throughout the subject's body, resulting in metastasis. Examples of tumor-associated mutations include but are not limited to mutations of the proto-oncogenes to become oncogenes.

The term “proto-oncogene” as used herein refers to a genetic sequence residing in the normal genome of a normal, non-tumor cell, which has the potential, when altered in the appropriate manner, of becoming an oncogene. The term “oncogene” as used herein refers to a gene, the aberrant expression or activity of which stimulates cell growth (e.g., abnormal cell growth). In some examples of the present disclosure, oncogenes encode for proteins that can be targeted by anti-cancer treatments. Examples of such proteins include but are not limited to, epidermal growth factor receptor (EGFR), serine/threonine-protein kinase (B-Raf), protein encoded by the KRAS proto-oncogene (K-Ras), echinoderm microtubule-associated protein-like 4-anaplastic lymphoma kinase (EML4-ALK), erb-b2 receptor tyrosine kinase 2 (ERBB2) and KIT proto-oncogene receptor tyrosine kinase (KIT). In one specific example, the protein is EGFR. In another specific example, the protein is KRAS. Examples of mutation mechanisms that caused the tumor-associated mutations include but are not limited to, substitution mutations, insertion mutations, inversion mutations, deletion mutations, gene rearrangement, gene fusions and frame shift mutations, as well as large-scale mutations in chromosomal structure.

In some examples, the mutations in the proteins expressed by the CTCs render the CTCs to be receptive to an anti-cancer treatment, while in some other examples, the mutations in the proteins expressed by the CTCs render the CTCs to be resistant to an anti-cancer treatment. Therefore, by detecting the mutations in the proteins expressed by the CTCs, the methods of the present disclosure allow the doctors to select the appropriate treatments for the cancer patients, and even to provide personalized treatments to the patients.

FIG. 8 illustrates an application of a method of the present disclosure for the selection of personalized treatment. In this example, a cancer patient carries a primary tumor with mutation profile A (MtA) and mutation profile B (MtB). The detection of CTCs using a method of the present disclosure indicates a higher level of MtA as a consequence of being the predominant tumor type, and a first tumor therapy is thus selected to target MtA first. After treatment using the first tumor therapy, the level of MtA decreased, and subsequently, a second tumor therapy is selected to target MtB. After this personalized course of treatment using the combination of the first and second tumor therapies, the person has a better chance of survival as the primary tumor carrying both types of mutations are prevented from metastasizing. In contrast, the current technologies for detecting CTCs based on the physical characteristics of CTCs or the expression of epithelial markers do not allow for the detection of different mutation profiles in the tumor, thus, the doctor may prescribe a tumor therapy that only targets one of the mutation profile (e.g. MtA). Since this tumor therapy will only be effective on tumor cells carrying MtA, tumor cells carrying MtB are left untreated, which may lead to metastasis of the primary tumor.

As used herein, the terms “treatment”, “treat”, “treating” or “amelioration” refers to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with a malignant condition or cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of a malignant disease, diminishment of extent of a malignant disease, stabilized (i.e., not worsening) state of a malignant disease, delay or slowing of progression of a malignant disease, amelioration or palliation of the malignant disease state, and remission (whether partial or total), whether detectable or undetectable. The term “treatment” of a disease also includes providing relief from the symptoms or side-effects of the disease (including palliative treatment). The term “anti-cancer treatment” should be construed accordingly. Examples of anti-cancer treatment include but are not limited to chemotherapy, radiotherapy, surgical treatment, immunotherapy and a combination thereof. In one specific example, the anti-cancer treatment comprises the use of an anti-cancer drug.

The term “receptive” means the anti-cancer treatment is effective on the cancer cells, in particular CTCs, of the patient. The term “resistant” means that the anti-cancer treatment is not effective on the cancer cells, in particular CTCs, of the patient.

Examples of mutations in EGFR that render the cancer cells, in particular CTCs, receptive to an anti-cancer treatment include but are not limited to, EGFR L858R, EGFR G719X, EGFR exon 19 (ΔE746-A750), EGFR V689M, EGFR N700D, EGFR E709K/Q, EGFR S720P, EGFR N826S, EGFR A839T, EGFR K846R, EGFR L861Q, EGFR G863D, EGFR V765A, EGFR T783A, EGFR exon 19 (ΔE746-T751), EGFR exon 19 (ΔE746-A750 (ins RP)), EGFR exon 19 (ΔE746-T751 (ins A/I)), EGFR exon 19 (ΔE746-T751 (ins VA)), EGFR exon 19 (ΔE746-S752 (ins A/V)), EGFR exon 19 (ΔL747-E749 (A750P)), EGFR exon 19 (ΔL747-A750 (ins P)), EGFR exon 19 (ΔL747-T751), EGFR exon 19 (ΔL747-T751 (ins P/S)), EGFR exon 19 (ΔL747-S752), EGFR exon 19 (ΔL747-752 (E746V)), EGFR exon 19 (ΔL747-752 (P753S)), EGFR exon 19 (ΔL747-S752 (ins Q)), EGFR exon 19 (ΔL747-P753), EGFR exon 19 (ΔL747-P753 (ins S)), EGFR exon 19 (ΔS752-I759). In some specific examples, the protein mutation EGFR L858R is resulting from mutation in the nucleic acid sequence EGFR coding DNA 2573 T>G; the protein mutation EGFR L861Q is resulting from mutation in the nucleic acid sequence EGFR coding DNA 2582 T>A; the protein mutation EGFR exon 19 (ΔE746-A750) is resulting from mutation in the nucleic acid sequence EGFR coding DNA 2235_2249 del15 GGAATTAAGAGAAGC; the protein mutation EGFR G719S is resulting from mutation in the nucleic acid sequence EGFR coding DNA 2155 G>A; the protein mutation EGFR G719C is resulting from mutation in the nucleic acid sequence EGFR coding DNA 2155 G>T; the protein mutation EGFR G719A is resulting from mutation in the nucleic acid sequence EGFR coding DNA 2155 G>C; the protein mutation EGFR exon 20 (ΔE746-A750) is resulting from mutation in the nucleic acid sequence EGFR coding DNA 2235_2249 del15 GGAATTAAGAGAAGC. Examples of mutations in EGFR that render the CTCs resistant to an anti-cancer treatment include but are not limited to, EGFR T790M, EGFR D761Y, EGFR exon 20 D770_N771 (ins NPG), EGFR exon 20 D770_N771 (ins SVQ), EGFR exon 20 D770_N771 (ins G), EGFR N771T, EGFR V769L and EGFR S768I. In some specific examples, the protein mutation EGFR T790M is resulting from mutation in the nucleic acid sequence EGFR coding DNA 2369 C>T; the protein mutation EGFR S768I is resulting from mutation in the nucleic acid sequence EGFR coding DNA 2303 G>T. For the above examples, the reference amino acid sequence for the wild-type EGFR is the sequence of SEQ ID NO: 1, and the reference nucleic acid sequence for the coding DNA of the wild-type EGFR gene is the sequence of SEQ ID NO: 2. In these examples, the anti-cancer treatment comprises the use of an anti-cancer drug such as a tyrosine kinase inhibitor or a monoclonal antibody against EGFR. Specific examples of tyrosine kinase inhibitors include but are not limited to afatinib, erlotinib, osimertinib and gefitinib. Examples of monoclonal antibodies against EGFR include but are not limited to cetuximab and panitumumab.

Examples of mutations in BRAF that render the cancer cells, in particular CTCs, receptive to an anti-cancer treatment include but are not limited to BRAF V600E and BRAF V600K. For the above examples, the reference amino acid sequence for the wild-type BRAF is the sequence of SEQ ID NO: 3. The reference nucleic acid sequence for the coding DNA of the wild-type BRAF gene is the sequence of SEQ ID NO: 8. In some examples, the anti-cancer treatment comprises the use of an anti-cancer drug such as a B-Raf inhibitor or a mitogen-activated protein kinase kinase (MEK) inhibitor. Specific examples of B-Raf inhibitors include but are not limited to dabrafenib and vemurafenib. Specific examples of MEK inhibitors include but are not limited to cobimetinib and trametinib.

Examples of mutations in KRAS that render the cancer cells, in particular CTCs, resistant to an anti-cancer treatment include but are not limited to KRAS codon 12 and KRAS codon 13 mutations. Examples of KRAS codon 12 mutations include but are not limited to KRAS G12V/C/S/D. An example of KRAS codon 13 mutation is KRAS G13D. In some specific examples, the protein mutation KRAS G12V is resulting from mutation in the nucleic acid sequence KRAS coding DNA 35 G>T; the protein mutation KRAS G12C is resulting from mutation in the nucleic acid sequence KRAS coding DNA 34 G>T; the protein mutation KRAS G12S is resulting from mutation in the nucleic acid sequence KRAS coding DNA 34 G>A; the protein mutation KRAS G12D is resulting from mutation in the nucleic acid sequence KRAS coding DNA 35 G>A; the protein mutation KRAS G13D is resulting from mutation in the nucleic acid sequence KRAS coding DNA 38 G>A. For the above examples, the reference amino acid sequence for the wild-type KRAS is the sequence of SEQ ID NO: 4, and the reference nucleic acid sequence for the coding DNA of the wild-type KRAS gene is the sequence of SEQ ID NO: 5. In some examples, the anti-cancer treatment comprises the use of an anti-cancer drug such as a monoclonal antibody against EGFR. Examples of monoclonal antibodies against EGFR include but are not limited to cetuximab and panitumumab.

Cancer cells, in particular CTCs, that are positive for ELM4-ALK fusion genes or proteins are generally receptive to ALK-targeted inhibitors. Thus, in some examples, the anti-cancer treatment for cancer cells, in particular CTCs, that are positive for ELM4-ALK fusion genes or proteins comprise the use of an anti-cancer drug such as ALK-targeted inhibitors. Specific examples of ALK-targeted inhibitors include but are not limited to alectinib, ceritinib and crizotinib.

Examples of mutations in ERBB2 that render the cancer cells, in particular CTCs, receptive to an anti-cancer treatment include but are not limited to ERBB2 V777L, D769H, D769Y and ERBB2 amplification. For the above examples, the reference amino acid sequence for the wild-type ERBB2 is the sequence of SEQ ID NO: 6. The reference nucleic acid sequence for the coding DNA of the wild-type ERBB2 gene is the sequence of SEQ ID NO: 9. In some examples, the anti-cancer treatment comprises the use of an anti-cancer drug such as an ERBB2 inhibitor or a monoclonal antibody against ERBB2. Specific examples of ERBB2 inhibitors include but are not limited to trastuzumab emtansine and lapatinib. Specific examples of monoclonal antibodies against ERBB2 include but are not limited to trastuzumab and pertuzumab.

An example of mutation in KIT that renders the cancer cells, in particular CTCs, resistance to an anti-cancer treatment is KIT V559D, KIT W557R and KIT L576P. For the above examples, the reference amino acid sequence for the wild-type KIT is the sequence of SEQ ID NO: 7. The reference nucleic acid sequence for the coding DNA of the wild-type KIT gene is the sequence of SEQ ID NO: 10. In one specific example, the anti-cancer treatment comprises the use of an anti-cancer drug such as a tyrosine-kinase inhibitor. One specific example of a tyrosine-kinase inhibitor is imatinib.

In one example of the present disclosure, there is provided a method of monitoring the progression of cancer, comprising determining the number of CTCs in a first sample obtained from the patient at an earlier time point and determining the number of CTCs in a second sample obtained from the patient at a later time point, wherein the presence of an increase in the number of CTCs in the second sample as compared to the first sample indicates that the cancer is progressing, and wherein the absence of an increase in the number of CTCs in the second sample as compared to the first sample indicates that the cancer is not progressing, and wherein the number of CTCs in the samples are determined using an antibody specifically binding to an antigen in a protein expressed by the CTCs, wherein the antigen comprises a mutation and wherein the mutation renders the patient receptive or resistant to the treatment with an anti-cancer drug. In some examples, the time difference between the early time point and the later time point is at least 1 week, or at least 2 weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 1 month, or at least 2 months, or at least 3 months, or at least 4 months, or at least 5 months, or at least 6 months, or at least 7 months, or at least 8 months, or at least 7 months, or at least 8 months, or at least 9 months, or at least 10 months, or at least 11 months, or at least 12 months, or at least 1 year, or at least 2 years, or at least 3 years, or at least 4 years, or at least 5 years. The time difference could also be determined by the number of treatment cycles. In some examples, the time difference between the early time point and the later time point is 1 treatment cycle, or 2 treatment cycles, or 3 treatment cycles, or 4 treatment cycles, or 5 treatment cycles, or 6 treatment cycles, or 7 treatment cycles, or 8 treatment cycles, or 9 treatment cycles, or 10 treatment cycles, or 11 treatment cycles, or 12 treatment cycles.

In some examples, it will be considered that an increase in the number of CTCs is present if the number of CTCs in the second sample as compared to the number of CTCs in the first sample has increased by at least 5%, or at least 10%, or at least 20%, or at least 30%, or at least 40%, or at least 50%, or at least 60%, or at least 70%, or at least 80%, or at least 90%, or at least 100%, or by at least 1 fold, or by at least 1.5 folds, or by at least 2 folds, or by at least 3 folds.

In another example of the present disclosure, there is also provided a method of monitoring and/or predicting the response to treatment of a cancer patient, comprising determining the number of CTCs in a sample obtained from the cancer patient before treatment and determining the number of CTCs in a sample obtained from the patient after treatment, wherein a reduction in the number of CTCs in the sample obtained after treatment as compared to the sample obtained before treatment indicates that the cancer patient is responding positively to the treatment, and wherein the number of CTCs in the samples are determined using an antibody specifically binding to an antigen in a protein expressed by the CTCs, wherein the antigen comprises a mutation and wherein the mutation renders the patient receptive or resistant to the treatment with an anti-cancer drug.

The term “reduction” or “reduced” grammatical variance refers to a decrease in the number of CTC in the test sample as compared to a control sample. In some examples, the number of CTCs in the test sample is reduced by at least 10%, at least 20%, at least 30%, at least 40%, at least 50%, at least 60%, at least 70%, at least 80%, at least 90% or at least 95% as compared to the number of CTCs in the control sample.

In another aspect of the present intention, there is provided a method of treating a cancer in a patient, wherein the method comprises: detecting circulating tumor cells (CTCs) in a sample obtained from the patient using an antibody specifically binding to an antigen in a protein expressed by the CTCs, wherein the antigen comprises a mutation and wherein the mutation renders the patient receptive or resistant to the treatment with an anti-cancer drug; and treating the patient with the anti-cancer drug suitable for the cancer to be treated in light of the results in (a).

In one example, the present disclosure provides a method of treating cancer in a patient, wherein the method comprises: detecting circulating tumor cells (CTCs) in a sample obtained from the patient using at least two antibodies specifically binding to at least two different antigens in one or more proteins expressed by the CTCs, wherein each of the at least two different antigens comprises at least one mutation and wherein the at least one mutation renders the patient receptive or resistant to the treatment with an anti-cancer drug, such that the detected CTCs comprise at least two different mutations that render the patient receptive or resistant to one or at least two different anti-cancer drugs, and treating the patient with one or at least two anti-cancer drugs suitable for the cancer to be treated in light of the results above. A person skilled in the art should be able to appreciate that when the CTCs detected in a sample of the patient carry one or more mutations that render the patient resistant to one or more particular anti-cancer drugs, those one or more anti-cancer drugs should not be selected for the treatment of the cancer patient. In one example, when at least two different anti-cancer drugs are used to treat the patient, these drugs are administered simultaneously. In another example, when at least two different anti-cancer drugs are used to treat the patient, these drugs are administered sequentially. When the at least two different anti-cancer drugs are administered sequentially, they can be administered immediately after each other, with a time difference in between, or in different treatment cycles. The time difference between each anti-cancer drug can be 1 hour, or 2 hours, or 3 hours, or 4 hours, or 5 hours, or 6 hours, or 7 hours, or 8 hours, or 9 hours, or 10 hours, or 11 hours, or 12 hours, or 15 hours, or 18 hours, or 21 hours, or 24 hours, or 1 day, or 2 days, or 3 days, or 4 days, or 5 days, or 6 days, or 7 days, or 8 days, or 9 days, or 10 days. When the at least two different anti-cancer drugs are used in different treatment cycles, the time difference between them could be 1 treatment cycle, or 2 treatment cycles, or 3 treatment cycles, or 4 treatment cycles, or 5 treatment cycles, or 6 treatment cycles, or 7 treatment cycles, or 8 treatment cycles, or 9 treatment cycles, or 10 treatment cycles, or 11 treatment cycles, or 12 treatment cycles. In some examples, the second anti-cancer drug is only administered after the treatment of the patient with the first anti-cancer drug is completed.

In some examples, the methods disclosed herein do not rely on physical characteristics (such as the size differences between CTCs and WBCs) for the detection of CTCs. In some other examples, the methods disclosed herein do not rely on epithelial markers (such as EPCAM and HER2) for the detection of CTCs. In some other examples, the methods disclosed herein do not rely on any of the above-mentioned physical characteristics or epithelial markers.

The major types of cancers or metastasizing forms of cancers that can be detected/diagnosed or treated by the methods as described herein include but are not limited to carcinoma, sarcoma, lymphoma, germ cell tumor and blastoma. The specific types of cancers that can be detected/diagnosed or treated by the method as described herein include but are not limited to lung cancer, melanoma, colorectal cancer, neuroblastoma, breast cancer, prostate cancer, renal cell cancer, transitional cell carcinoma, cholangiocarcinoma, brain cancer, non-small cell lung cancer, pancreatic cancer, gastric carcinoma, bladder cancer, esophageal cancer, mesothelioma, thyroid cancer, head and neck cancer, osteosarcoma, hepatocellular carcinoma, carcinoma of unknown primary, ovarian carcinoma, endometrial carcinoma, glioblastoma, Hodgkin lymphoma and non-Hodgkin lymphomas. In some specific examples, the cancers to be detected/diagnosed or treated by the method disclosed herein are lung cancer, melanoma, colorectal cancer, lymphoma and neuroblastoma. In one specific example, the cancer to be detected/diagnosed or treated by the method disclosed herein is lung cancer. In another specific example, the cancer to be detected/diagnosed or treated by the method disclosed herein is colorectal cancer.

In one example, the cancer is invasive and/or metastatic cancer. In another example, the cancer is stage II cancer, stage III cancer or stage IV cancer. In another example, the cancer is an early metastatic cancer. In one example, early metastatic cancer is not necessarily an early stage cancer.

As used herein, the singular form “a”, “an” and “the” include plural references unless the context clearly dictates otherwise. For example, the term “a primer” includes a plurality of primers, including mixtures thereof.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain examples may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain examples may also be described broadly and generically herein. Each of the narrower species and subgeneric groupings falling within the generic disclosure also form part of the disclosure. This includes the generic description of the examples with a proviso or negative limitation removing any subject matter from the genus, regardless of whether or not the excised material is specifically recited herein.

### EXPERIMENTAL SECTION

### Example 1—Testing Specificity of Mutation-Specific Antibodies

In order to test the specificity of the antibodies, Western blot analysis was carried out using EGFR L858R and EGFR exon19 (ΔE746-A750) antibodies on H1975 (with known EGFR L858R mutation) and PC9 (with known EGFR exon19 (ΔE746-A750) mutation), and using KRAS G12V antibody on SW620 (with known KRAS G12V mutation) and PC9 (known as negative for KRAS-G12V), according to the following protocol:

1. The cell lysate from PC9, H1975 and SW620 cancer lines were harvested using RIPA buffer added with protease inhibitor and incubated at 4° C. for 30 minutes.

2. The lysate was sonicated for three times for 30 seconds.

3. The protein was denatured at 99° C. for 5 minutes in laemmli buffer.

4. Equal amount of samples were loaded to the polyacrylamide gel and electrophoresis was performed at constant 20 mA for 1 hour in Tris Glycine SDS running buffer.

5. The protein was transferred to polyvinylidene difluoride (PVDF) membrane in cold Tris-Glycine transfer buffer for 1 hour.

6. The membrane was incubated with blocking buffer (5% skimmed milk in TBS buffer added with 0.1% Tween 20, TBST) for 1 hour at room temperature, followed by two 10 minutes washings with TBST.

7. The membrane was incubated with primary antibody (1:500 dilution) at 4° C. overnight, followed by four 10 minutes washings with TBST.

8. The membrane was incubated with secondary antibody (1:200 dilution) for 1 hour at room temperature, followed by four 10 minutes washings with TBST.

9. The membrane was added with ECL substrate and imaged with chemiluminescence imaging system.

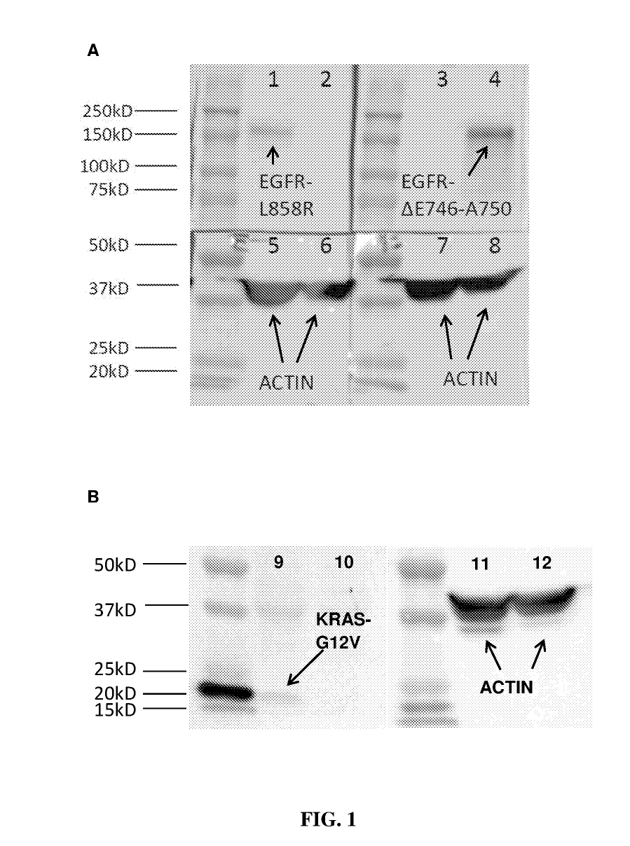

The results shown in FIG. 1A indicate that the EGFR L858R and EGFR exon19 (ΔE746-A750) antibodies can detect the EGFR mutations with good specificity, as positive signal for EGFR L858R was only detected in the sample from H1975 cells (Lane 1) and positive signal for EGFR exon19 (ΔE746-A750) was only detected in the sample from PC9 cells (Lane 4). Similarly, the results shown in FIG. 1B indicate that the KRAS G12V antibody can detect the KRAS G12V mutation with good specificity, as positive signal for KRAS G12V was only detected in the sample from SW620 cells (Land 9).

### Example 2—Detecting Tumor Cells Using Mutation-Specific Antibodies

Immuno-fluorescent experiment was carried out to detect tumor cells in the samples using mutation-specific antibodies EGFR L858R and EGFR exon19 (ΔE746-A750) according to the following protocol:

1. Cytospin was performed on approximately 100,000 cells.

2. The cells were incubated with FcR blocking reagent for 15 mins at 4° C. followed by CD45 staining for 30 minutes at 4° C.

3. The cells were washed for three times followed permeabilization with 0.1% Triton-X for 10 mins at room temperature.

4. The blocking buffer supplemented with goat serum was added to the sample and incubate for 1 hour.

5. Primary antibody was added to the sample followed by overnight incubation at 4° C.

6. Secondary antibody was added to the sample followed by 1 hour incubation at room temperature.

7. DAPI stain was added to the sample and the slide was mounted with coverslip before visualization on the microscope.

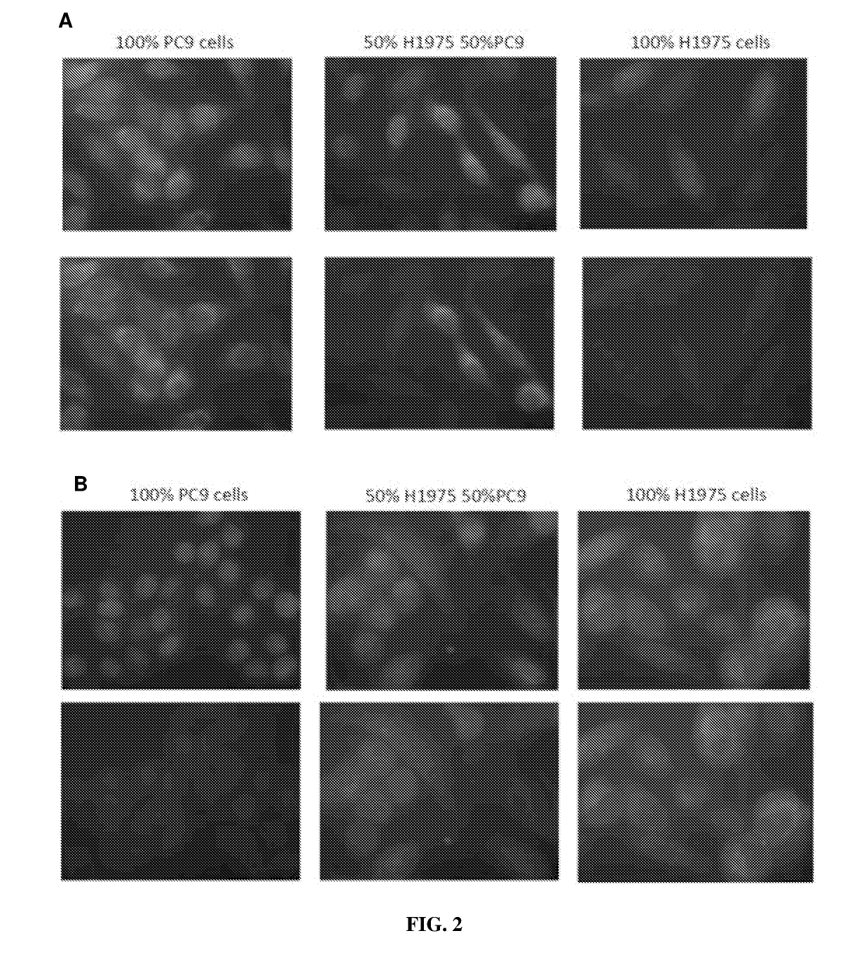

A series of experiments using different percentages of H1975 and PC9 cells were carried out. The results in FIG. 2 show that positive signals were only observed in cells that carry the mutation signature targeted by the mutation-specific antibody.

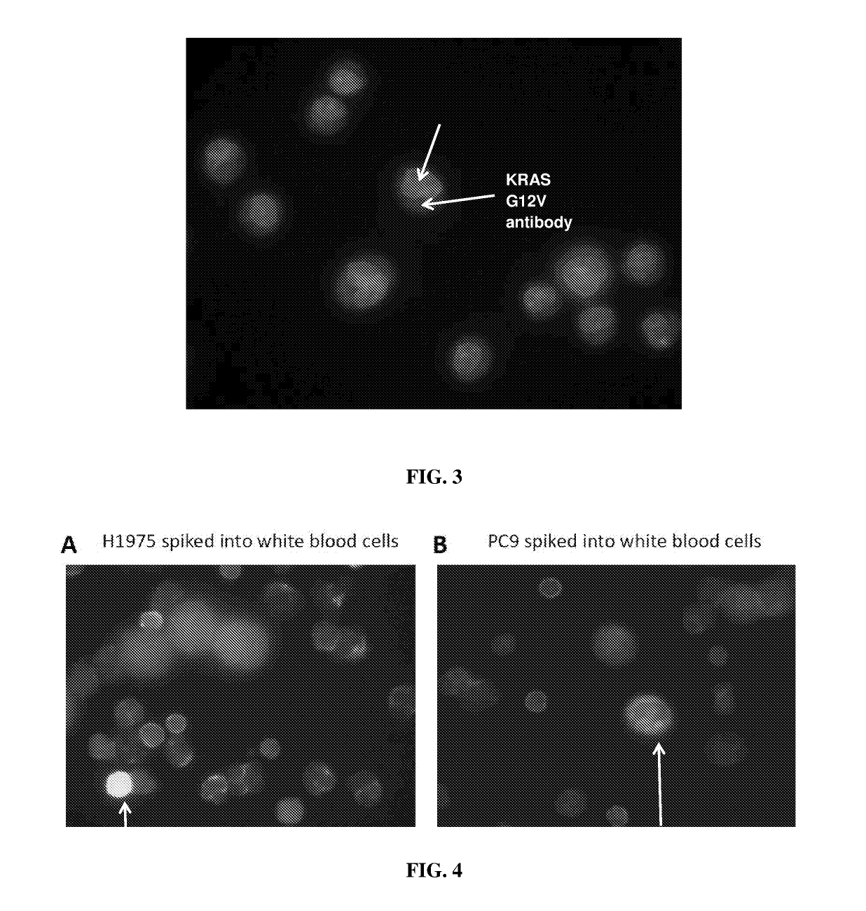

Using the same immuno-fluorescent protocol above, experiment using 100% SW620 cells was carried out for KRAS G12V antibody. The results in FIG. 3 show that positive signals were observed in all SW620 cells, confirming the sensitivity of the KRAS-G12V antibody.

### Example 3—Detecting Tumor Cells in Blood Samples from Healthy Subjects Spiked with Tumor Cells

To simulation samples containing CTCs, blood samples from healthy subjects were collected and spiked with cancer cells H1975 or PC9. The ratio of the spiked cancer cells and the healthy blood cells is 1:9 (10% of cancer cells and 90% of healthy blood cells). The cells were mixed gently and immunofluorescence assay was carried out according to the following protocol:

1. Cytospin was performed on approximately 100,000 cells.

2. The cells were incubated with FcR blocking reagent for 15 mins at 4° C. followed by CD45 staining for 30 minutes at 4° C.

3. The cells were washed for three times followed permeabilization with 0.1% Triton-X for 10 mins at room temperature.

4. The blocking buffer supplemented with goat serum was added to the sample and incubate for 1 hour.

5. Primary antibody was added to the sample followed by overnight incubation at 4° C.

6. Secondary antibody was added to the sample followed by 1 hour incubation at room temperature.

7. DAPI stain was added to the sample and the slide was mounted with coverslip before visualization on the microscope.

In order to distinguish the cancer cells from the white blood cells (WBCs) in the blood samples, CD45 antibody was also introduced. CD45 negative indicates that the cells are cancer cells, while CD45 positive indicates that the cells are WBCs. The results in FIG. 4 show that positive signals of EGFR L858R were only observed for H1975 cells and positive signals of EGFR exon19 (ΔE746-A750) were only observed for PC9 cells.

### Example 4—Detecting Tumor Cells in Clinical Samples

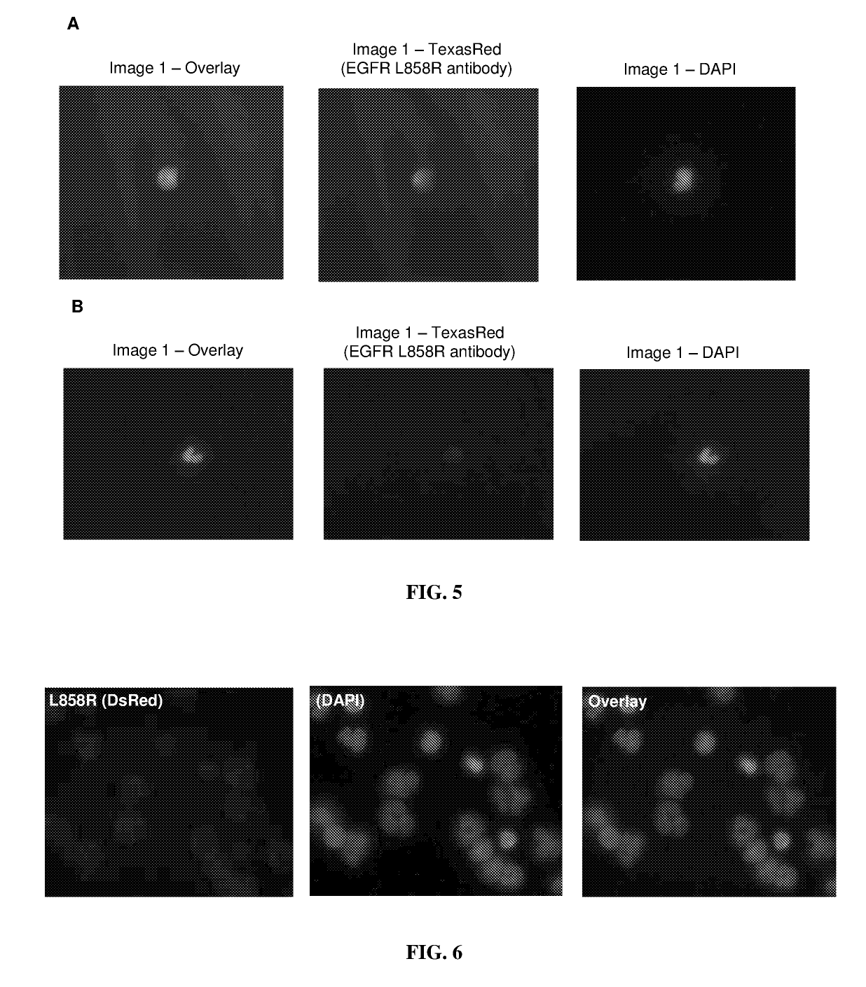

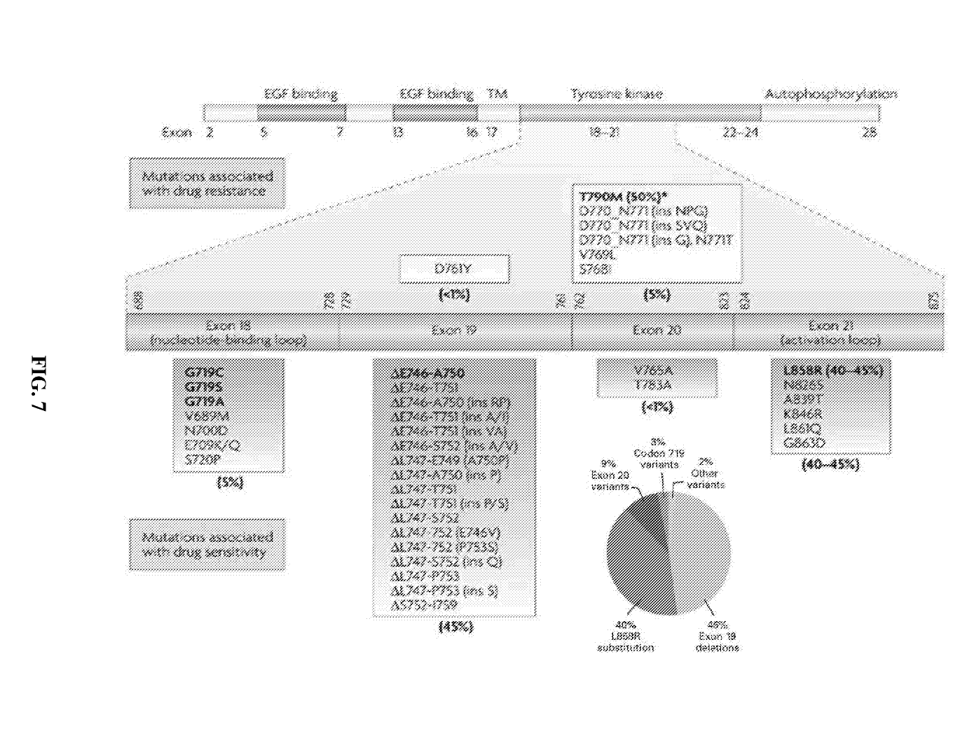

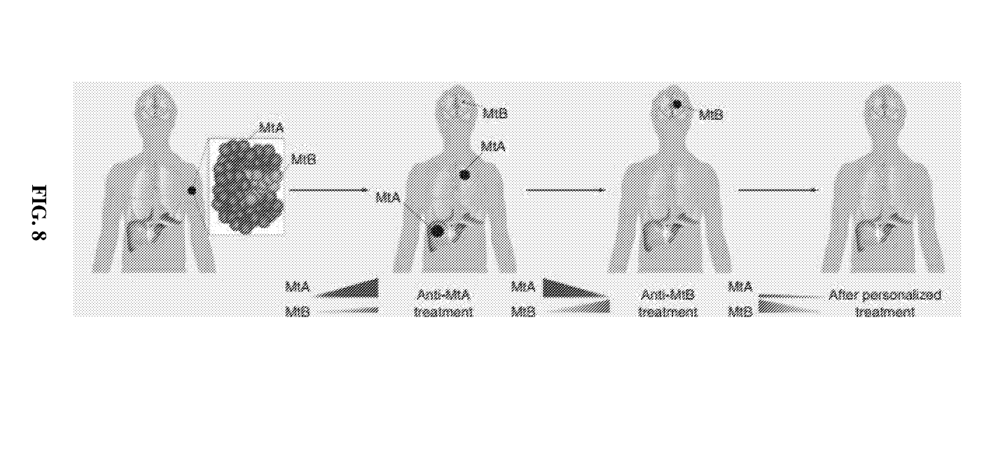

To test the sensitivity of the antibodies in clinical settings, immuno-fluorescent experiments were carried out using blood samples obtained from lung cancer patient with positive EGFR L858R mutation in the primary tumor. Details of the patient are as follows: Stage 1A, T1N0M0, tumor size=1.9×1.7×1.5 cm, primary tumor was tested positive with EGFR L858R mutation. Blood samples obtained from healthy subjects were used as the negative control. As shown in FIG. 6, the cells in the blood samples obtained from healthy subjects were shown to be negative for EGFR L858R signals, indicating the absence of CTCs. As shown in FIG. 5, the immuno-fluorescent results of blood samples from lung cancer patients with positive EGFR L858R mutation in the primary tumor were shown to be positive for EGFR L858R, indicating the presence of CTCs.

1. A method of detecting and/or diagnosing cancer in a patient, wherein the method comprises detecting circulating tumor cells (CTCs) in a sample obtained from the patient using an antibody specifically binding to an antigen in a protein expressed by the CTCs, wherein the antigen comprises a mutation and wherein the mutation renders the patient receptive or resistant to an anti-cancer treatment.

2. The method of claim 1, wherein the method of detecting and/or diagnosing comprises monitoring the progression of cancer, comprising determining the number of CTCs in a first sample obtained from the patient at an earlier time point and determining the number of CTCs in a second sample obtained from the patient at a later time point, wherein the presence of an increase in the number of CTCs in the second sample as compared to the first sample indicates that the cancer is progressing, and wherein the absence of an increase in the number of CTCs in the second sample as compared to the first sample indicates that the cancer is not progressing.

3. The method of claim 1, wherein the protein is encoded by a proto-oncogene.

4. The method of claim 3, wherein the protein encoded by the proto-oncogene is selected from the group consisting of EGFR, B-Raf, K-Ras, ALK-EML4, ERBB2 and KIT.

5. The method of claim 4, wherein the protein is EGFR or K-Ras.

6. The method of claim 5, wherein the mutation in EGFR is selected from the group consisting of L858R, G719X, exon 19 (ΔE746-A750), L861Q, T790M and exon 20 insertion.

7. The method of claim 5, wherein the mutation in K-Ras is selected from the group consisting of G12V, G12C, G12S, G12D and G13D.

8. The method of claim 5, wherein the mutation in B-Raf is V600E or V600K.

9. The method of claim 5, wherein the mutation in KIT is selected from the group consisting of V559D, W557R and L576P.

10. A method of treating a cancer in a patient, wherein the method comprises:
(a) detecting circulating tumor cells (CTCs) in a sample obtained from the patient using an antibody specifically binding to an antigen in a protein expressed by the CTCs, wherein the antigen comprises a mutation and wherein the mutation renders the patient receptive or resistant to an anti-cancer treatment; and
(b) treating the patient with the anti-cancer treatment suitable for the cancer to be treated in light of the results in (a).

11. The method of claim 10, wherein the anti-cancer treatment comprises a treatment selected from the group consisting of chemotherapy, radiotherapy, surgical treatment, immunotherapy and a combination thereof.

12. The method of claim 11, wherein the anti-cancer treatment comprises chemotherapy.

13. The method of claim 9, wherein the protein is encoded by a proto-oncogene.

14. The method of claim 13, wherein the protein encoded by a proto-oncogene is selected from the group consisting of EGFR, B-Raf, K-Ras, ALK-EML4, ERBB2 and KIT.

15. The method of claim 14, wherein the protein is EGFR.

16. The method of claim 15, wherein the mutation in EGFR is selected from the group consisting of L858R, G719X, exon 19 (ΔE746-A750) and L8610.

17. (canceled)

18. The method of claim 1, wherein the cancer is selected from the group consisting of carcinoma, sarcoma, lymphoma, germ cell tumor, blastoma, lung cancer, melanoma, colorectal cancer, neuroblastoma, breast cancer, prostate cancer, renal cell cancer, transitional cell carcinoma, cholangiocarcinoma, brain cancer, non-small cell lung cancer, pancreatic cancer, gastric carcinoma, bladder cancer, esophageal cancer, mesothelioma, thyroid cancer, head and neck cancer, osteosarcoma, hepatocellular carcinoma, carcinoma of unknown primary, ovarian carcinoma, endometrial carcinoma, glioblastoma, Hodgkin lymphoma and non-Hodgkin lymphomas.

19. (canceled)

20. A method of detecting and/or diagnosing lung cancer in a patient, wherein the method comprises detecting circulating tumor cells (CTCs) in a sample obtained from a patient using an antibody specifically binding to an antigen in a protein expressed by the CTCs, wherein the antigen comprises a mutation of EGFR L858R or EGFR exon 19 (ΔE746-A750), and wherein the mutation renders the patient receptive to the treatment with a tyrosine kinase inhibitor.

21. A method of detecting and/or diagnosing colorectal cancer in a patient, wherein the method comprises detecting circulating tumor cells (CTCs) in a sample obtained from a patient using an antibody specifically binding to an antigen in a protein expressed by the CTCs, wherein the antigen comprises a mutation of K-Ras selected from the group consisting of K-Ras G12V, K-Ras G12C, K-Ras G12S, K-Ras G12D and K-Ras G13D, and wherein the mutation renders the patient resistant to the treatment with a monoclonal antibody against EGFR.

22. (canceled)

23. The method of claim 1, wherein detecting circulating tumor cells (CTCs) in a sample further comprises distinguishing white blood cells (WBCs) from CTCs using at least one WBC marker.

24. (canceled)


SEQUENCE LISTING (10 sequences)

SEQ ID NO: 1 - protein, 1210 aa, Homo sapiens [sequence omitted]

SEQ ID NO: 2 - DNA, 3633 bp, Homo sapiens
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 3 - protein, 766 aa, Homo sapiens [sequence omitted]

SEQ ID NO: 4 - protein, 188 aa, Homo sapiens [sequence omitted]

SEQ ID NO: 5 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 6 - protein, 1255 aa, Homo sapiens [sequence omitted]

SEQ ID NO: 7 - protein, 976 aa, Homo sapiens [sequence omitted]

SEQ ID NO: 8 - DNA, 2301 bp, Homo sapiens
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactga

SEQ ID NO: 9 - DNA, 3768 bp, Homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtga

SEQ ID NO: 10 - DNA, 2931 bp, Homo sapiens
atgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctga